(19) United States
(12) Patent Application Publication
(10) Pub. No.: US 2019/0388328 A1
(43) Pub. Date: Dec. 26, 2019

(54) COSMETIC COMPOSITION FOR TEMPORARILY SHAPING KERATINIC FIBERS, BASED ON RENEWABLE RAW MATERIALS

(71) Applicant: Henkel AG & Co. KGaA, Duesseldorf (DE)

(72) Inventors: Marcus Noll, Quickborn (DE); Marie Borson, Hamburg (DE)

(73) Assignee: Henkel AG & Co. KGaA, Duesseldorf (DE)

(21) Appl. No.: 16/433,644

(22) Filed: Jun. 6, 2019

(30) Foreign Application Priority Data
Jun. 25, 2018 (DE) 10 2018 115 212.4

(51) Int. Cl. A61K 8/81; A61K 8/73; A61Q 5/06

(52) U.S. Cl. CPC A61K 8/8129; A61Q 5/06; A61K 8/73

(57) ABSTRACT

A cosmetic composition for temporarily shaping keratinous fibers, in particular human hair, is provided. The cosmetic composition comprises a film-forming agent comprising a homopolymer or copolymer derived from itaconic acid and a rheology modifier comprising a polysaccharide. A method for temporarily shaping keratinous fibers using the cosmetic composition also is provided.

### CROSS-REFERENCE TO RELATED APPLICATION

This application claims priority to German Patent Application No. 10 2018 115 212.4, filed Jun. 25, 2018, which is incorporated herein by reference in its entirety.

### TECHNICAL FIELD

The present disclosure relates to cosmetic compositions for temporarily shaping keratinic fibers based on renewable raw materials, comprising a film-forming agent and a rheology modifier. Furthermore, the present disclosure relates to the use of the cosmetic compositions as contemplated herein, as well as to a method for temporarily shaping keratinic fibers using the cosmetic compositions as contemplated herein.

### BACKGROUND

Cosmetic agents which serve to permanently or temporarily style the hair have an important role in cosmetics. Temporary styling, which should provide a good hold without compromising the healthy appearance of the hair, such as its shine, for example, can be obtained by using hair sprays, hair waxes, hair gels, hair mousses, etc., for example. The cosmetic agents are blended as a function of these forms of applications.

An important property of an agent for temporarily shaping keratinic fibers is that the treated fibers should be endowed with as strong a hold as possible in the shape which is produced. When the keratinic fibers are human hair, this is also known as a strong styling hold or a high degree of hold of the cosmetic agent. The styling hold is essentially determined by the type and quantity of polymers used.

Many cosmetic agents which ensure temporary styling are known in the prior art. Appropriate agents usually contain synthetic polymers as the styling components. Thus, what are known as film-forming agents constitute polymeric components which are used for the purposes of temporary styling. Cosmetic preparations often also contain rheology modifiers as further polymeric components in order to thicken the cosmetic preparations, so that the cosmetic preparations can be handled more easily. Conventional thickening polymers, however, are more and more becoming the focus of criticism in the context of discussions regarding microplastics because they have unforeseeable effects on the environment into which they are introduced.

The polymers which are usually used in agents for temporary styling are manufactured from corresponding synthetically accessible monomers. The monomers in turn are obtained from fossil materials such as, for example, crude oil by conversion into the corresponding polymer building blocks, expending energy, inter alia, in the process. Because even the monomers are produced synthetically, the polymers which are usually employed are fully synthetic polymers. In the context of sustainable management of raw materials and energy, there is still a desire to use only those cosmetic raw materials in cosmetic products which are accessible from renewable raw materials with as little energy expenditure as possible. More and more customers want cosmetic products which are free from petrochemicals. A reduction in quantity or even exchanging fully synthetic polymers can, however, only be undertaken if the replacement polymers have the desired properties for the application and if they provide the keratinic fibers with a sufficient, stable hold of the set style. Furthermore, the use of a plurality of alternative polymers must be both mutually compatible and compatible with other ingredients of the cosmetic agent.

### BRIEF SUMMARY

In accordance with an exemplary embodiment, a cosmetic composition for temporarily shaping keratinous fibers is provided. The cosmetic composition comprises a film-forming agent and a rheology modifier, wherein the film-forming agent comprises a homopolymer or copolymer derived from itaconic acid, having formula (I):

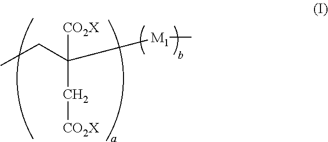

in which the repeat unit M₁ represents a vinyl monomer unit, X represents hydrogen or an alkali metal, a represents the molar proportion of the repeat unit derived from itaconic acid in the homopolymer or copolymer and b represents the molar proportion of the repeat unit M₁ in the homopolymer or copolymer, wherein a is from about 50 mol % to about 100 mol % and b is from about 0 mol % to about 50 mol %, and wherein the rheology modifier comprises a polysaccharide.

In accordance with another exemplary embodiment, a method for temporarily shaping keratinous fibers is provided. The method comprises the steps of applying to the keratinous fibers a cosmetic composition comprising:

a film-forming agent and a rheology modifier, wherein the film-forming agent comprises a homopolymer or copolymer derived from itaconic acid, having formula (I):

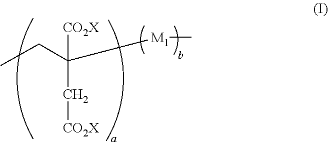

in which the repeat unit M₁ represents a vinyl monomer unit, X represents hydrogen or an alkali metal, a represents the molar proportion of the repeat unit derived from itaconic acid in the homopolymer or copolymer and b represents the molar proportion of the repeat unit M₁ in the homopolymer or copolymer, wherein a is from about 50 mol % to about 100 mol % and b is from about 0 mol % to about 50 mol %, and wherein the rheology modifier comprises a polysaccharide; and subsequently shaping the keratinous fibers.

In accordance with a further exemplary embodiment, a cosmetic composition for temporarily shaping keratinous fibers is provided. The cosmetic composition comprises: about 2 to about 15% by weight of a film-forming agent based on the total weight of the cosmetic composition, wherein the film-forming agent comprises a homopolymer or copolymer derived from itaconic acid, having formula (I):

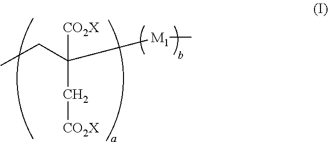

in which the repeat unit M₁ represents a vinyl monomer unit, X represents hydrogen or an alkali metal, a represents the molar proportion of the repeat unit derived from itaconic acid in the homopolymer or copolymer and b represents the molar proportion of the repeat unit M₁ in the homopolymer or copolymer, wherein a is from about 50 mol % to about 100 mol % and b is from about 0 mol % to about 50 mol %, and from about 0.1 to about 5% by weight of a rheology modifier based on the total weight of the cosmetic composition, wherein the rheology modifier comprises a polysaccharide, wherein the polysaccharide comprises an organically modified natural substance based on a cellulose or the polysaccharide comprises a naturally occurring hydrocolloid.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

An objective of the present disclosure includes providing a cosmetic composition which is suitable for temporarily shaping keratinic fibers, in which the use of fully synthetic polymers and/or of polymers of petrochemical origin is significantly reduced or wherein the use of these polymers is dispensed with, wherein the properties of the cosmetic composition remain the same to a substantial extent. In particular, the polymers as well as the rheology modifiers endowing the hair with the stable hold should not be fully synthetic polymers or polymers of petrochemical origin.

The objective of the present disclosure is achieved by employing the subject matter of claim 1. In a first aspect, therefore, the present disclosure concerns a cosmetic composition for temporarily shaping keratinous fibers, in particular human hair, comprising a film-forming agent and a rheology modifier, wherein the film-forming agent is a homopolymer or copolymer derived from itaconic acid, having formula (I):

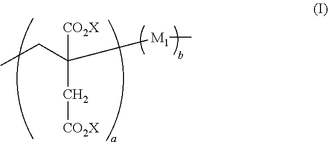

in which the repeat unit M₁ represents a vinyl monomer unit, X represents hydrogen or an alkali metal, a represents the molar proportion of the repeat unit derived from itaconic acid in the homopolymer or copolymer and b represents the molar proportion of the repeat unit M₁ in the homopolymer or copolymer, wherein a is from about 50 mol % to about 100 mol % and b is from about 0 mol % to about 50 mol ° and wherein the rheology modifier is a polysaccharide.

In accordance with the present disclosure, the cosmetic composition comprises a film-forming agent. The term “film-forming agent” should be understood to mean those polymers which, upon drying, leave a continuous film on the skin, the hair or the nails. Film-forming agents of this type may be used in very different cosmetic products such as, for example, face masks, make-up, hair setting lotions, hair sprays, hair gels, hair waxes, hair treatments, shampoos or nail polishes. Polymers of the type which have a sufficient solubility in water, alcohol or water/alcohol mixtures are preferred. Appropriate solutions can be produced in this manner, which can be used or processed further in a simple manner.

In the context of the present disclosure, the term “film-forming agent” should in particular be understood to mean those polymers which, when used in from about 0.05 to about 20 percent by weight (with respect to the total weight of the cosmetic agent) aqueous, alcoholic or aqueous-alcoholic solution, are capable of being deposited on the hair in the form of a transparent polymer film.

As contemplated herein, the film-forming agent is a homopolymer or copolymer which is derived from itaconic acid. When the film-forming agent is exclusively in the form of polymerized itaconic acid and/or a salt of itaconic acid, the film-forming agent forms a homopolymer.

The technical production of itaconic acid is conventionally carried out biotechnologically by submerged fermentation of molasses and other substrates using strains of specific fungi. In this manner, the monomer for the production of the film-forming agent constitutes a raw material which is not produced fully synthetically and which does not derive directly from petrochemical sources. In the context of the present disclosure, a film-forming agent would then be considered to have been produced fully synthetically if all the monomers constituting the film-forming agent were derived conventionally, directly and synthetically from petrochemical sources. The advantage with the use of polymers from itaconic acid as a film-forming agent lies in the conservation of resources.

Alternatively, the film-forming agent is a copolymer which is constructed from polymerized itaconic acid and/or from a salt of itaconic acid and a further monomer, which forms the monomer unit M₁ in the copolymer. As contemplated herein, the monomer unit M₁ is a vinyl monomer unit, i.e. the monomer unit M₁ is formed from monomers which have a vinyl function which, following polymerization, become part of the backbone of the copolymer. The advantage with the use of such polymers for cosmetic compositions as contemplated herein lies in an improved possibility for controlling the properties of the polymers purposefully for the use in cosmetic compositions. In fact, most vinyl monomers are of conventional petrochemical origin, however as contemplated herein, the fraction of the monomer units M₁ in the copolymer is preferably smaller than the fraction of monomer units which are derived from itaconic acid.

The copolymer having formula (I) may be a block copolymer or a random copolymer. Preferably, the copolymer having formula (I) is a random copolymer. The variables a and b are a measure of the proportion of monomer units in the copolymers used as contemplated herein; a and b are normalized to about 100 mol %. In other words, the degree of polymerization of the copolymer used as contemplated herein is with respect to about 100 mol %. When, for example in a copolymer the degree of polymerization is about 100 and the number average for the monomer derived from itaconic acid is about 60 and the monomer unit M₁ is about 30, then a is about 60 mol % and b is about 30 mol %. In addition to the monomer unit derived from itaconic acid and the monomer unit M₁, in preferred embodiments, other monomer units may be contained in the polymer. Thus, the film-forming agent used as contemplated herein may also be terpolymers.

The monomer units derived from itaconic acid may be present as the free acid, and then X is equal to hydrogen, or they may be present as salts of the acid, in which case X represents an alkali metal, for example sodium. Examples of a particularly preferred substance is the product “RevCare NE 100S”™ from Itaconix LLC. This may also be pre-neutralized.

The cosmetic composition contains a rheology modifier as a further essential component. The term “rheology modifier” as used in the context of the present disclosure should be understood to mean a thickening agent. The thickening agent is used for the purposes of increasing the viscosity of the cosmetic composition. In general, thickening agents are substances which alter the flow properties of fluids in a manner such that fluids with a thickening agent have a higher viscosity than without thickening agent. In the context of the present disclosure, a preferred rheology modifier is a polymer which, when added to the cosmetic composition in quantities of from about 0.05 to about 10% by weight with respect to the total weight of the cosmetic composition, results in an increase in the viscosity. The rheology modifier is strictly different from the film-forming agent.

In accordance with a preferred embodiment of the present disclosure, the film-forming agent of the cosmetic composition is a copolymer derived from itaconic acid, having formula (II):

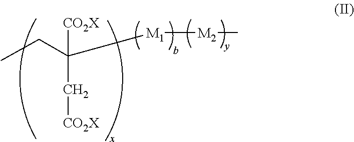

as a decarboxylation product of the homopolymer or copolymer derived from itaconic acid having formula (I), in which the repeat unit M₂ is a second repeat unit derived from itaconic acid which is obtained by employing decarboxylation of the monomer unit derived from itaconic acid, wherein the sum of x and y is equal to a and y>0 mol %.

The homopolymer or copolymer having formula (I) may undergo a decarboxylation. Formally, at least one carbon dioxide molecule is removed from the monomer unit derived from itaconic acid. In this manner, a copolymer having formula (II) is obtained. This can occur by heating or by enzymatic reaction. The monomer unit M₂ is obtained from a monomer unit derived from itaconic acid in this manner.

The copolymer having formula (II) may be a block copolymer in which the monomer unit derived from itaconic acid and the monomer unit M₂ are randomly distributed in a block next to the block formed from M₁, or the copolymer having formula (II) may be a random copolymer. Preferably, the copolymer having formula (II) is a random copolymer.

In accordance with a preferred embodiment of the present disclosure, the film-forming agent in the cosmetic composition is the homopolymer or copolymer having formula (I) or the copolymer having formula (II), in which a is from about 60 to about 100 mol %, preferably from about 70 to about 98 mol %, more preferably from about 80 to about 95 mol %, most preferably from about 85 to about 90 mol %; and/or the film-forming agent is the homopolymer or copolymer having formula (I) or the copolymer having formula (II), in which b is from about 0 to about 40 mol %, preferably from about 2 to about 30 mol %, more preferably from about 5 to about 20 mol %, most preferably from about 10 to about 15 mol %, and/or the film-forming agent is the copolymer having formula (II), in which y is from about 1 to about 100 mol %, preferably from about 10 to about 90 mol %, more preferably from about 50 to about 80 mol %, most preferably from about 60 to about 70 mol %.

In the context of the present disclosure, the proportions of the co-monomers in the film-forming copolymer, i.e. the variables a, b, x and v in the copolymers having formula (I) and (II), are deter mined by employing NMR analysis.

Homopolymers or copolymers of these preferred embodiments are particularly suitable for the use in cosmetic compositions.

The cosmetic composition contains a rheology modifier as the second mandatory component. In accordance with a preferred embodiment of the present disclosure, the polysaccharide in the cosmetic composition as contemplated herein is an organically modified natural substance based on cellulose, preferably hydroxyethylcellulose, hydroxypropylcellulose or microcrystalline cellulose. While hydroxy ethylcellulose and hydroxypropylcellulose constitute components which are modified natural substances, microcrystalline cellulose is a natural substance which originates in its entirety from non-petrochemical sources. Examples of commercially available products of the preferably employed polysaccharides are “Tylose H 100000 YP2”™ from SE Tylose GmbH, or “KluCel HCS”™ from Ashland.

Microcrystalline cellulose is obtained from lignified plant parts, in which the non-crystalline cellulose fractions are separated by using an acidic medium at temperatures of over 100° C. The microcrystalline cellulose may optionally be carboxylated. Hydroxyethylcellulose, hydroxypropylcellulose and microcrystalline cellulose constitute excellent rheology-modifiers for using in cosmetic agents.

Alternatively, the polysaccharide in accordance with a preferred embodiment is a naturally occurring hydrocolloid; preferably xanthan gum; gum Arabic, in particular gum Arabic derived from the plant species Acacia Senegal, Acacia Seyal, Acacia Karroo and Acacia Laeta; gellan gum; carrageenan; carob bean gum, in particular tara gum; or it is a starch, in particular a starch derived from the plant species maize, wheat or Oryza Sativa rice.

A “hydrocolloid” is a technological abbreviation for the more correct specialist description “hydrophilic colloid”. Hydrocolloids are macromolecules which preferably are largely linear in shape and have intermolecular interacting forces which enable a physical network to be formed. These water-soluble polymers constitute a large group of chemically very different natural and synthetic polymers the common features of which are their solubility in water or aqueous media and the possibility of the formation of physical networks. The prerequisite for this is that these polymers possess a sufficient number of hydrophilic groups to be soluble in water and that they are not chemically cross-linked to too great an extent. The hydrophilic groups may be of a non-ionic, anionic or cationic nature.

The preferred hydrocolloids all derive from natural sources and in this manner have the advantage that the components of the cosmetic compositions are obtained in a manner which conserves resources and energy. Furthermore, the entry of microplastics into the environment is completely avoided. Examples of commercially available products with the preferred hydrocolloids which are used as contemplated herein are “Amaze XT”™ from AkzoNobel, “Solagum Tara”™ from Seppic, “FMC AC 150”™ from FMC Biopolymer AS, “Remy DR-KA”™ from Argana, “GENUVISCO CG 131”™ from CP Kelco, “KELCOGEL CG HA”™ from CP Kelco.

In accordance with a further preferred embodiment of the present disclosure, in the cosmetic composition, the repeat unit M₁ is derived from alkyl acrylates or alkyl methacrylates, in particular methyl(meth)acrylate, from alkyl itaconates, in particular dimethyl itaconate or diethyl itaconate, from maleic acid anhydrides, styrene, acrylonitrile, vinyl halides, vinyl alcohols, vinyl acetate/vinyl alcohol mixtures, olefins or mixtures thereof.

By introducing co-monomer units into the polymer forming the film-forming agent, its properties can be adjusted. Thus, styrene groups make the copolymer rather more hydrophobic, while vinyl alcohol when used as the co-monomer render the copolymer rather more hydrophilic.

In accordance with a further preferred embodiment, in the cosmetic composition, the homopolymer or copolymer having formula (I) or the decarboxylation product formed from the copolymer having formula (II) has a degree of neutralization of from about 35 mol % to about 65 mol %, preferably from about 40 mol % to about 60 mol %, more preferably from about 45 mol % to about 55 mol %. The degree of neutralization is a measure of the proportion of free acid groups in the homopolymer or copolymer. The total proportion of carboxyl groups in the homopolymer or copolymer should be about 100 mol %. The degree of neutralization provides the proportion of free acid groups in the molecule. Homopolymers or copolymers of this preferred embodiment are advantageously in cosmetic agents.

In accordance with a further preferred embodiment of the present disclosure, the film-forming agent is contained in the cosmetic composition in a quantity of from about 2 to about 15% by weight, preferably in a quantity of from about 3 to about 10% by weight, more preferably in a quantity of from about 4% to about 7% by weight, with respect to the total weight of the cosmetic composition.

If the film-forming agents used as contemplated herein are employed in a quantity of less than about 2% by weight, then, although a film is formed on the keratinous fibers, the layer thickness of the films which are formed are too low, so that the styling hold is not strong enough. If the quantity of film-forming agent exceeds about 15% by weight, this could result in the unwanted formation of flakes.

In accordance with a further preferred embodiment of the present disclosure, the rheology modifier is contained in the cosmetic composition in a quantity of from about 0.1 to about 5% by weight, preferably in a quantity of from about 0.2 to about 3% by weight, more preferably in a quantity of from about 0.3 to about 2.5% by weight, with respect to the total weight of the cosmetic composition.

In principle, the quantity of rheology modifier is a function of the desired viscosity of the cosmetic agent and on the type of rheology modifier. In particular, when selecting the preferred rheology modifier used as contemplated herein, the preferred ranges for the quantities are intended to produce viscosities which result in products which satisfy customer expectations in a particularly good manner.

In accordance with a further preferred embodiment, the cosmetic composition is water-based. Preferably, the cosmetic composition has a water content of from about 50 to about 97% by weight, more preferably from about 75 to about 96% by weight, most preferably from about 85 to about 95% by weight, with respect to the total weight of the cosmetic composition. Alternatively, the cosmetic composition comprises an aqueous-alcoholic support, wherein preferably, the aqueous-alcoholic support contains a C1 to C4 alcohol, in particular ethanol or isopropanol. A water content which is as high as possible has the advantage of being kind to the skin of the user of the cosmetic agent. If, in contrast, an aqueous-alcoholic support is used, then the components, in particular the polymeric components, are rendered more compatible with each other.

In accordance with a further preferred embodiment, the cosmetic composition contains a plant extract, preferably a plant extract from bamboo, linseed, lotus, green tea, oak bark, stinging nettle, hamamelis, hops, henna, camomile, burdock root, horsetail, whitethom, lime blossom, almond, aloe vera, spruce needle, horse chestnut, sandalwood, juniper, coconut, mango, apricot, lime, wheat, kiwi, melon, orange, grapefruit, sage, rosemary, birch, mallow, cuckoo flower, wild thyme, yarrow, thyme, melissa, harrow, coltsfoot, mallow, meristem, ginseng and ginger root. The plant extracts endow the cosmetic composition with a pleasant fragrance on the one hand. On the other hand, they act as preservatives. Particularly in the case in which the preservative phenyl propanol is used, the plant extracts produce a mild but sufficient preservative action. Thus, in a particularly preferred embodiment, the cosmetic composition contains the plant extracts cited above and phenyl propanol.

More particularly preferred cosmetic compositions as contemplated herein comprise at least one of the following embodiments A) to F):

A)

A cosmetic composition for temporarily shaping keratinous fibers, in particular human hair, comprising a film-forming agent and a rheology modifier, wherein the film-forming agent is a homopolymer or copolymer derived from itaconic acid, having formula (I):

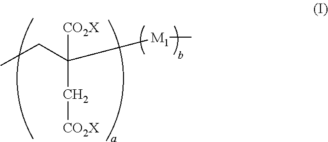

in which the repeat unit M₁ represents a vinyl monomer unit, X represents hydrogen or an alkali metal, a represents the molar proportion of the repeat unit derived from itaconic acid in the homopolymer or copolymer and b represents the molar proportion of the repeat unit M₁ in the homopolymer or copolymer, wherein a is from about 50 mol % to about 100 mol % and b is from 0 mol % to about 50 mol %, and wherein the rheology modifier is a polysaccharide which is selected from hydroxyethylcellulose, hydroxypropyl cellulose or microcrystalline cellulose.

B)

A cosmetic composition for temporarily shaping keratinous fibers, in particular human hair, comprising a film-forming agent and a rheology modifier, wherein the film-forming agent is a homopolymer or copolymer derived from itaconic acid, having formula (I):

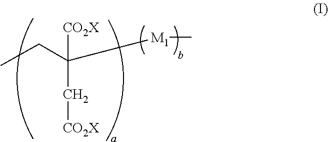

in which the repeat unit M₁ represents a vinyl monomer unit, X represents hydrogen or an alkali metal, a represents the molar proportion of the repeat unit derived from itaconic acid in the homopolymer or copolymer and b represents the molar proportion of the repeat unit M1 in the homopolymer or copolymer, wherein a is from about 50 mol % to about 100 mol % and b is from 0 mol % to about 50 mol %, and wherein the rheology modifier is selected from xanthan gum; gum Arabicum, in particular gum Arabicum derived from the plant species Acacia Senegal, Acacia Seyal, Acacia Karroo and Acacia Laeta; gellan gum; carrageenan; carob bean gum, in particular tara gum; or starch, in particular starch derived from the plant varieties corn, wheat or Oryza Sativa rice.

C)

A more particularly preferred cosmetic composition in accordance with embodiment A or B, wherein a is from about 60 to about 100 mol %, preferably from about 70 to about 98 mol %, more preferably from about 80 to about 95 mol %, most preferably from about 85 to about 90 mol %, and/or wherein b is from 0 to about 40 mol %, preferably from about 2 to about 30 mol %, more preferably from about 5 to about 20 mol %, most preferably from about 10 to about 15 mol %.

D)

A cosmetic composition for temporarily shaping keratinous fibers, in particular human hair, comprising a film-forming agent and a rheology modifier, wherein the film-forming agent is a homopolymer or copolymer derived from itaconic acid, having formula (I):

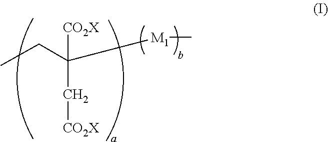

in which the repeat unit M₁ represents a vinyl monomer unit, X represents hydrogen or an alkali metal, a represents the molar proportion of the repeat unit derived from itaconic acid in the homopolymer or copolymer and b represents the molar proportion of the repeat unit M1 in the homopolymer or copolymer, wherein a is from about 50 mol % to about 100 mol % and b is from 0 mol % to about 50 mol %, or wherein the film-forming agent is a copolymer derived from itaconic acid having formula (II):

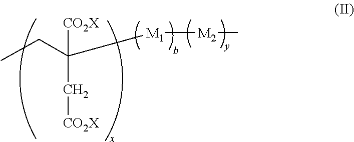

as a decarboxylation product of the homopolymer or copolymer derived from itaconic acid having formula (I), in which the repeat unit M₂ is a second repeat unit derived from itaconic acid, which is obtained by employing a decarboxylation of the repeat unit derived from itaconic acid, wherein the sum of x and y is equal to a and y>0 mol %, wherein the repeat unit M₁ is derived from alkyl acrylates or alkyl methacrylates, in particular methyl(meth)acrylate, from alkyl itaconates, in particular dimethyl itaconate or ethyl itaconate, from maleic acid anhydrides, styrene, acrylonitrile, vinyl halides, vinyl alcohols, vinyl acetate/vinyl alcohol mixtures, olefins or mixtures thereof.

E)

A cosmetic composition for temporarily shaping keratinous fibers, in particular human hair, comprising a film-forming agent and a rheology modifier, wherein the film-forming agent is a homopolymer or copolymer derived from itaconic acid, having formula (I):

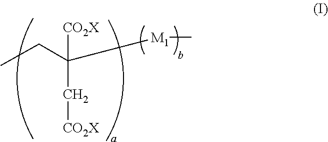

in which the repeat unit M₁ represents a vinyl monomer unit, X represents hydrogen or an alkali metal, a represents the molar proportion of the repeat unit derived from itaconic acid in the homopolymer or copolymer and b represents the molar proportion of the repeat unit M1 in the homopolymer or copolymer, wherein a is from about 50 mol % to about 100 mol % and b is from 0 mol % to about 50 mol %, and wherein the rheology modifier is a polysaccharide, which is selected from hydroxyethylcellulose, hydroxypropylcellulose or microcrystalline cellulose, or wherein the rheology modifier is selected from xanthan gum; gum Arabicum, in particular gum Arabicum derived from the plant species Acacia Senegal, Acacia Seyal, Acacia Karroo and Acacia Laeta; gellan gum; carrageenan; carob bean gum, in particular tara gum; or starch, in particular starch derived from the plant varieties corn, wheat or Oryza Sativa rice; wherein the film-forming agent is contained in the cosmetic composition in a quantity of from about 2 to about 15% by weight, preferably in a quantity of from about 3 to about 10% by weight, more preferably in a quantity of from about 4 to about 7% by weight, with respect to the total weight of the cosmetic composition, and/or wherein the rheology modifier is contained in the cosmetic composition in a quantity of from about 0.1 to about 5% by weight, preferably in a quantity of from about 0.2 to about 3% by weight, more preferably in a quantity of from about 0.3 to about 2.5% by weight, with respect to the total weight of the cosmetic composition.

F)

Furthermore, the cosmetic compositions of the more particularly preferred embodiments A to E are more particularly preferably used for temporarily shaping human hair.

The use of the cosmetic composition of the first aspect of the present disclosure for temporarily shaping and/or fixing the style of keratinous fibers, in particular human hair, constitutes a second aspect of the present disclosure.

In accordance with a preferred embodiment of the present disclosure, the cosmetic composition is used as a hair spray, hair wax, hair gel, hair treatment, hair setting lotion, shampoo, non-aerosol pump spray, aerosol mousse, non-aerosol mousse, styling gel, pomade, lotion, or cream.

A method for temporarily shaping keratinous fibers, in particular human hair, constitutes a third aspect of the present disclosure, wherein the cosmetic composition of the first aspect of the present disclosure is applied to the keratinous fibers.

In particular, the third aspect of the present disclosure concerns a method for temporarily shaping keratinous fibers, in particular human hair, in which in a first step, a cosmetic composition as contemplated herein is applied to the keratinous fibers, in particular to human hair, and in a subsequent step, the keratinous fibers, in particular the human hair, are shaped. As an alternative, in a first step, a cosmetic composition as contemplated herein is applied to the keratinous fibers, in particular to human hair, in the subsequent step at least part of the cosmetic composition is rinsed out and the keratinous fibers, in particular human hair, are then shaped. As contemplated herein, the keratinous fibers may then be dried if necessary.

Features relating to preferred embodiments of the first aspect of the present disclosure which are only described above in respect thereof are clearly also applicable to the second aspect and the third aspect as features of preferred embodiments.

The examples below are intended to illustrate the subject matter of the present disclosure without in any way limiting it.

### EXAMPLES

The following examples in accordance with the cosmetic compositions as contemplated herein could be produced:

1^{st} Composition

| | | Quantity | |
|---|---|---|---|
| Component | Trademark | (in wt. %) | Function |
| Sodium itaconate | RevCare NE 100S | 4.0 | Film-forming |
| | (from Revolymer) | | agent |
| Phenyl propanol and | Conarom B | 1.0 | Preservative |
| Humulus Lupulus hop | (from Ashland) | | |
| extract | | | |
| Dehydroxyxanthan | Amaze XT | 1.2 | Rheology |
| gum | (from Akzo) | | modifier |
| Water | | Ad 100 | Support/solvent |

2^{nd} Composition

| | | Quantity | |
|---|---|---|---|
| Component | Trade mark | (in wt. %) | Function |
| Sodium itaconate | RevCare NE 100S | 4.5 | Film-forming |
| | | | agent |
| Phenyl propanol and | Conarom B | 1.0 | Preservative |
| Humulus Lupulus hop | | | |
| extract | | | |
| Tara gum | Solagum Tara | 1.8 | Rheology |
| | (from Seppic) | | modifier |
| Water | | Ad 100 | Support/solvent |

3^{rd} Composition

| | | Quantity | |
|---|---|---|---|
| Component | Trade mark | (in wt. %) | Function |
| Sodium itaconate | RevCare NE 100S | 6.0 | Film-forming |
| | | | agent |
| Phenyl propanol and | Conarom B | 1.0 | Preservative |
| Humulus Lupulus hop | | | |
| extract | | | |
| Acacia Senegal gum | FMC AC 150 | 0.6 | Rheology |
| | (from FMC | | modifier |
| | biopolymer AS) | | |
| Water | | Ad 100 | Support/solvent |

4^{th} Composition

| | | Quantity | |
|---|---|---|---|
| Component | Trade mark | (in wt. %) | Function |
| Sodium itaconate | RevCare NE 100S | 4.0 | Film-forming |
| | | | agent |
| Phenyl propanol and | Conarom B | 1.0 | Preservative |
| Humulus Lupulus hop | | | |
| extract | | | |
| Corn Starch Modified | Amaze (from Akzo) | 0.75 | Rheology |
| | | | modifier |
| Water | | Ad 100 | Support/solvent |

5^{th} Composition

| | | Quantity | |
|---|---|---|---|
| Component | Trade mark | (in wt. %) | Function |
| Sodium itaconate | RevCare NE 100S | 3.2 | Film-forming |
| | | | agent |
| Phenyl propanol and | Conarom B | 1.0 | Preservative |
| Humulus Lupulus hop | | | |
| extract | | | |
| Triticum Vulgare | Wheat Starch | 2.5 | Rheology |
| (Wheat) Starch | (from Merck) | | modifier |
| Water | | Ad 100 | Support/solvent |

6^{th} Composition

| | | Quantity | |
|---|---|---|---|
| Component | Trade mark | (in wt. %) | Function |
| Sodium itaconate | RevCare NE 100S | 3.2 | Film-forming |
| | | | agent |
| Phenyl propanol and | Conarom B | 1.0 | Preservative |
| Humulus Lupulus hop | | | |
| extract | | | |
| Oryza Sativa (Rice) | Remy DR-KA | 1.6 | Rheology |
| Starch | (from Agrana) | | modifier |
| Water | | Ad 100 | Support/solvent |

7^{th} Composition

| | | Quantity | |
|---|---|---|---|
| Component | Trade mark | (in wt. %) | Function |
| Sodium itaconate | RevCare NE 100S | 6.5 | Film-forming |
| | | | agent |
| Phenyl propanol and | Conarom B | 1.0 | Preservative |
| Humulus Lupulus hop | | | |
| extract | | | |
| Carrageenan | Genuvisco CG 131 | 0.6 | Rheology |
| | (from CP Kelco) | | modifier |
| Water | | Ad 100 | Support/solvent |

8^{th} Composition

| | | Quantity | |
|---|---|---|---|
| Component | Trade mark | (in wt. %) | Function |
| Sodium itaconate | RevCare NE 100S | 6.5 | Film-forming |
| | | | agent |
| Phenyl propanol and | Conarom B | 1.0 | Preservative |
| Humulus Lupulus hop | | | |
| extract | | | |
| Gellan gum | Kelcogel CG HA | 0.15 | Rheology |
| | (from CP Kelco) | | modifier |
| Water | | Ad 100 | Support/solvent |

9^{th} Composition

| | | Quantity | |
|---|---|---|---|
| Component | Trade mark | (in wt. %) | Function |
| Sodium itaconate | RevCareNE 100S | 4.0 | Film-forming |
| | | | agent |
| Phenyl propanol and | Conarom B | 1.0 | Preservative |
| Humulus Lupulus hop | | | |
| extract | | | |
| Hydroxyethylcellulose | Tylose H 100000 | 2.0 | Rheology |
| | YP2 (from SE | | modifier |
| | Tylose GmbH) | | |
| Water | | Ad 100 | Support/solvent |

10^{th} Composition

| | | Quantity | |
|---|---|---|---|
| Component | Trade mark | (in wt. %) | Function |
| Sodium itaconate | RevCareNE 100S | 4.0 | Film-forming |
| | | | agent |
| Phenyl propanol and | Conarom B | 1.0 | Preservative |
| Humulus Lupulus hop | | | |
| extract | | | |
| Hydroxypropylcellulose | KluCel HCS | | Rheology |
| | (from Ashland) | | modifier |
| Water | | Ad 100 | Support/solvent |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A cosmetic composition for temporarily shaping keratinous fibers comprising a film-forming agent and a rheology modifier, wherein the film-forming agent comprises a homopolymer or copolymer derived from itaconic acid, having formula (I): 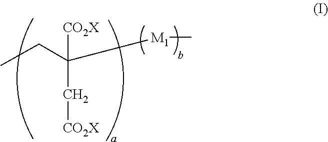
in which the repeat unit M₁ represents a vinyl monomer unit, X represents hydrogen or an alkali metal, a represents the molar proportion of the repeat unit derived from itaconic acid in the homopolymer or copolymer and b represents the molar proportion of the repeat unit M₁ in the homopolymer or copolymer, wherein a is from about 50 mol % to about 100 mol % and h is from about 0 mol % to about 50 mol %, and wherein the rheology modifier comprises a polysaccharide.

2. The cosmetic composition as claimed in claim 1, wherein the film-forming agent of the cosmetic composition comprises a copolymer derived from itaconic acid, having formula (II): 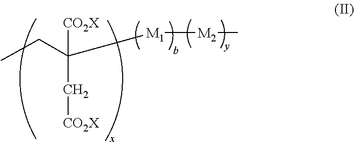
as a decarboxylation product of the homopolymer or copolymer derived from itaconic acid, having formula (I), in which the repeat unit M₂ is a second repeat unit derived from itaconic acid which is obtained by means of decarboxylation of the monomer unit derived from itaconic acid, wherein the sum of x and y is equal to a and y>0 mol %.

3. The cosmetic composition as claimed in claim 2, wherein the film-forming agent comprises the homopolymer or copolymer having formula (I) or the copolymer having formula (II), in which a is from about 85 to about 90 mol %,

4. The cosmetic composition as claimed in claim 1, wherein the polysaccharide comprises an organically modified natural substance based on a cellulose, or wherein the polysaccharide is a naturally occurring hydrocolloid.

5. The cosmetic composition as claimed in claim 1, wherein the repeat unit M₁ is derived from alkyl acrylates or alkyl methacrylates, alkyl itaconates, maleic acid anhydrides, styrene, acrylonitrile, vinyl halides, vinyl alcohols, vinyl acetate/vinyl alcohol mixtures, olefins or mixtures thereof.

6. The cosmetic composition as claimed in claim 1, wherein the film-forming agent is included in the cosmetic composition in a quantity of from about 2 to about 15% by weight with respect to the total weight of the cosmetic composition and the rheology modifier is included in the cosmetic composition in a quantity of from about 0.1 to about 5% by weight with respect to the total weight of the cosmetic composition.

7. The cosmetic composition as claimed in claim 1, wherein the cosmetic composition comprises water in the amount of about 50 to about 97 by weight with respect to the total weight of the cosmetic composition.

8. The cosmetic composition as claimed in claim 1, wherein the homopolymer or copolymer having formula (I) or the decarboxylation product from the copolymer having formula (II) has a degree of neutralization of from about 35 mol % to about 65 mol %.

9. A method for temporarily shaping keratinous fibers, the method comprising the steps of:
applying to the keratinous fibers a cosmetic composition comprising:
a film-forming agent and a rheology modifier, wherein the film-forming agent comprises a homopolymer or copolymer derived from itaconic acid, having formula (I): 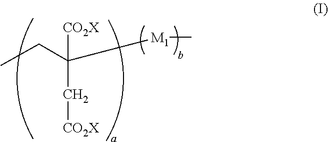
in which the repeat unit M₁ represents a vinyl monomer unit, X represents hydrogen or an alkali metal, a represents the molar proportion of the repeat unit derived from itaconic acid in the homopolymer or copolymer and b represents the molar proportion of the repeat unit M₁ in the homopolymer or copolymer, wherein a is from about 50 mol % to about 100 mol % and b is from about 0 mol % to about 50 mol %, and wherein the rheology modifier comprises a polysaccharide; and
subsequently shaping the keratinous fibers.

10. The method as claimed in claim 9, wherein after applying but before the shaping, at least a portion of the cosmetic composition is rinsed from the keratinous fibers.

11. The cosmetic composition as claimed in claim 2, wherein the film-forming agent comprises the homopolymer or copolymer having formula (I) or the copolymer having formula (II), in which b is from about 10 to about 15 mol %.

12. The cosmetic composition as claimed in claim 2, wherein the film-forming agent comprises the copolymer having formula (II), in which y is from about 1 to 100 mol %.

13. The cosmetic composition as claimed in claim 4, wherein the cellulose is selected from the group comprising hydroxyethylcellulose, hydroxypropylcellulose or microcrystalline cellulose.

14. The cosmetic composition as claimed in claim 4, wherein the hydrocolloid is selected from the group comprising xanthan gum, gum Arabic, gellan gum, carrageenan, carob bean gum, and starch.

15. The cosmetic composition as claimed in claim 1, wherein the cosmetic composition comprises an aqueous-alcoholic support that comprises a C1 to C4 alcohol.

16. The method as claimed in claim 9, wherein the film-forming agent of the cosmetic composition comprises a copolymer derived from itaconic acid, having formula (II): 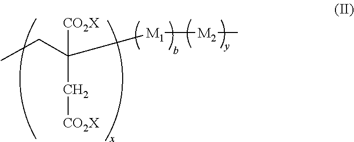
as a decarboxylation product of the homopolymer or copolymer derived from itaconic acid, having formula (I), in which the repeat unit M₂ is a second repeat unit derived from itaconic acid which is obtained by means of decarboxylation of the monomer unit derived from itaconic acid, wherein the sum of x and y is equal to a and y>0 mol %.

17. The method as claimed in claim 9, wherein the polysaccharide comprises an organically modified natural substance based on a cellulose, or wherein the polysaccharide comprises a naturally occurring hydrocolloid.

18. The method as claimed in claim 9, wherein the repeat unit M₁ is derived from alkyl acrylates or alkyl methacrylates, alkyl itaconates, maleic acid anhydrides, styrene, acrylonitrile, vinyl halides, vinyl alcohols, vinyl acetate/vinyl alcohol mixtures, olefins or mixtures thereof.

19. The method as claimed in claim 9, wherein the film-forming agent s included in the cosmetic composition in a quantity of from about 2 to about 15% by weight with respect to the total weight of the cosmetic composition and the rheology modifier is included in the cosmetic composition in a quantity of from about 0.1 to about 5% by weight with respect to the total weight of the cosmetic composition.

20. A cosmetic composition for temporarily shaping keratinous fibers, comprising:
about 2 to about 15% by weight of a film-forming agent based on the total weight of the cosmetic composition, wherein the film-forming agent comprises a homopolymer or copolymer derived from itaconic acid, having formula (I): 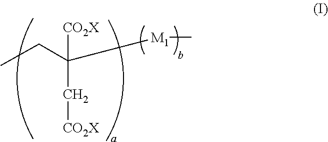
in which the repeat unit M₁ represents a vinyl monomer unit, X represents hydrogen or an alkali metal, a represents the molar proportion of the repeat unit derived from itaconic acid in the homopolymer or copolymer and b represents the molar proportion of the repeat unit M₁ in the homopolymer or copolymer, wherein a is from about 50 mol % to about 100 mol % and b is from about 0 mol % to about 50 mol %, and
from about 0.1 to about 5% by weight of a rheology modifier based on the total weight of the cosmetic composition, wherein the rheology modifier comprises a polysaccharide,
wherein the polysaccharide comprises an organically modified natural substance based on a cellulose or the polysaccharide comprises a naturally occurring hydrocolloid.
